(19) United States
(12) Patent Application Publication
(10) Pub. No.: US 2009/0005839 A1
(43) Pub. Date: Jan. 1, 2009

(54) Skin Tanning System Incorporating Skin Rejuvenating Light

(76) Inventors: Roy Lloyd Griffith, Indianapolis, IN (US); Gregory Richard Bender, Carmel, IN (US); Matthew Lee Adams, Indianapolis, IN (US); Randall Jay Ballentine, Indianapolis, IN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/088,895

(22) PCT Filed: Jun. 29, 2006
(86) PCT No.: PCT/US06/25366

§ 371 Date: Jul. 9, 2008

Related U.S. Application Data
(60) Provisional application No. 60/723,553, filed on Oct. 4, 2005.

(51) Int. Cl. A61N 5/06

(52) U.S. Cl. 607/91

(57) ABSTRACT

In a preferred embodiment the present invention provides a tanning system with base unit and canopy unit mounted to define a gap for a person to be tanned. The canopy unit defines a facial area to be before the person's face. The facial area provides ultraviolet illumination coupled with illumination in the visible light and/or infrared light spectrums. In one embodiment, the system includes one or more facial tanners and one or more skin rejuvenation portions mounted in the facial area. Preferably, the facial area includes one skin rejuvenation portion mounted in the center of the facial area before the person with a face to be tanned and two facial tanners mounted laterally beside the skin rejuvenation portion within the facial area.

### CROSS REFERENCE TO RELATED APPLICATIONS

The present application is a national stage of International Patent Application PCT/US2006/025366 filed Jun. 29, 2006, which claims the benefit of U.S. Provisional Application No. 60/723,553 filed Oct. 4, 2005, all of which are hereby incorporated by reference.

### FIELD OF THE INVENTION

This invention relates to ultraviolet tanning. A novel combination of a body tanning system and a facial area light source can be suitable for providing ultraviolet illumination in selective combination with specific visible and/or infrared illumination.

### BACKGROUND OF THE INVENTION

The most common method of artificial skin tanning involves the process of exposing skin to ultraviolet light. Arrangements of ultraviolet lamps have been used for, among other things, tanning beds, tanning booths, face tanners, and tanning canopies. The present invention relates to a combination of the benefits of exposure to ultraviolet light during tanning with the exposure of the facial area to certain types of visible and/or infrared light.

Many types of tanning systems have two or more portions which are moveable between an open position and a closed position for the ease of access of the user. When the portions are in the operating position, a volume or gap is defined between them where the person to be tanned or treated is oriented. During treatment, the user's skin receives ultraviolet illumination emitted by the unit. Some units in the prior art have light emitting diode (LED) displays located near the user's head, so as to be visible during the tanning session, which can display timing information or other information concerning the operation of the bed. These LED displays contain a limited number of LEDs (less than 30) which are believed to be continuously operating, with a relatively low intensity, to serve as status lights and/or collectively as numeric digits, such as may display the minutes remaining on the timer for the unit.

### SUMMARY OF THE INVENTION

An illustration of a preferred embodiment of the present invention is a tanning system. The tanning system includes a first unit and a second unit mounted to be positioned in an operating position with respect to the first unit such that the first and second units define a gap for a person, having a face to be tanned, to be oriented. In the case of a tanning bed having a lower bench and an upper canopy, the canopy defines a facial area before the face of the person to be tanned. The facial area is operable to selectively provide visible and/or infrared illumination in pre-determined predominant wavelengths towards the face of the person to be tanned.

In an alternate embodiment, the present invention provides a system for tanning having a first unit mounted to a second unit, and a facial mask for selectively providing visible and infrared illumination in pre-determined predominant wavelengths.

In yet another embodiment, the present invention provides a system for tanning a person. The system includes a device defining a location for a person with a face to be tanned; and a facial area associated with the tanning device oriented to be before the face of the person to be tanned. The facial unit is operable to selectively provide ultraviolet illumination in combination with certain visible and infrared light in pre-determined predominant wavelengths towards the face of the person to be tanned.

This summary is provided to introduce a selection of concepts in a simplified form that are described in further detail in the detailed description and drawings contained herein. This summary is not intended to identify key features or essential features of the claimed subject matter, nor is it intended to be used as an aid in determining the scope of the claimed subject matter. Yet other forms, embodiments, objects, advantages, benefits, features, and aspects of the present invention will become apparent from the detailed description and drawings contained herein.

### BRIEF DESCRIPTION OF THE DRAWINGS

FIG. 1 is a perspective view of a tanning bed according to one preferred embodiment of the present invention.

FIG. 2 is a partial enlarged view of one end of the tanning bed of FIG. 1.

FIG. 3 is a partial enlarged view of one end of a tanning bed according to an alternate embodiment of the present invention.

FIG. 4 is an alternate perspective view of the tanning bed of FIG. 1.

FIG. 5 is a perspective view of a tanning bed according to an alternate embodiment of the present invention.

FIG. 6 is a perspective view of a tanning booth according to yet another alternate embodiment of the present invention.

FIG. 7 is a functional view of a tanning system according to a further preferred embodiment of the present invention.

### DESCRIPTION OF ILLUSTRATIVE EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described device, and any further applications of the principles of the invention as described herein are contemplated as would normally occur to one skilled in the art to which the invention relates.

Traditionally, ultraviolet illumination/radiation systems are available in a variety of configurations, including beds, booths, canopies, and facial systems. These systems are often used by people to achieve the cosmetic affect of suntanned looking skin, or to obtain medical benefits such as satisfying the body's vitamin D requirements or treating psoriasis. For purposes of explaining the illustrative embodiments, a tanning bed and tanning booth are described in detail with reference to a “person with a face to be tanned.”

In addition to tanning the body, it may also be desirable to expose the face of a person to certain wavelengths of visible and/or infrared illumination. This process is commonly known as “phototherapy.” Research is being conducted by others that may indicate that exposure to specific wavelengths in the electromagnetic spectrum can stimulate the production of collagen, reduce swelling, relieve pain, reduce the presence of wrinkles, inhibit the presence of acne, and otherwise promote skin wellness.

The electromagnetic spectrum can be broken down into classes including infrared, visible, and ultraviolet light. These sub classifications are typically broken down into wavelength ranges of 1 mm-750 nm for infrared light, 750 nm-400 nm for visible light, and 400 nm-10 nm for ultraviolet light.

It is also indicated by recent research by others that the diverse tissue and cell types in the body all have their own unique light absorption characteristics. Additionally, some chemical reactions are also influenced by specific frequencies or ranges of frequencies of light. Therefore, certain parts of the skin respond to light at a specific wavelength while others do not. For example, visible red light of wavelength 660 nm is believed to penetrate to a depth of 8-10 mm, making it beneficial in treating problems close to the skin's surface, while infrared light of wavelength 904 nm is believed to penetrate to a depth of 30-40 mm, which would make it more effective in promoting deep skin and muscle wellness. Additionally, visible light in the range of about 415 nm has been shown to kill bacteria in the pores which eventually lead to the development of acne. It is also believed that the effects of the illumination, in some cases, are enhanced when the source of the illumination is pulsed.

References herein to “visible illumination” are intended to mean the emission of specific, predetermined radiation wavelengths predominantly in the visible light spectrum desired for phototherapy, and are not references to ultraviolet illumination, although ultraviolet illumination could theoretically also be present as an incidental component. Similarly, references herein to “infrared illumination” are intended to mean the emission of specific, predetermined radiation wavelengths predominantly in the infrared light spectrum desired for phototherapy. A “non-UV” light source is intended to mean a light source that is principally providing visible or infrared illumination without a substantial UV component, although incidental UV might be present.

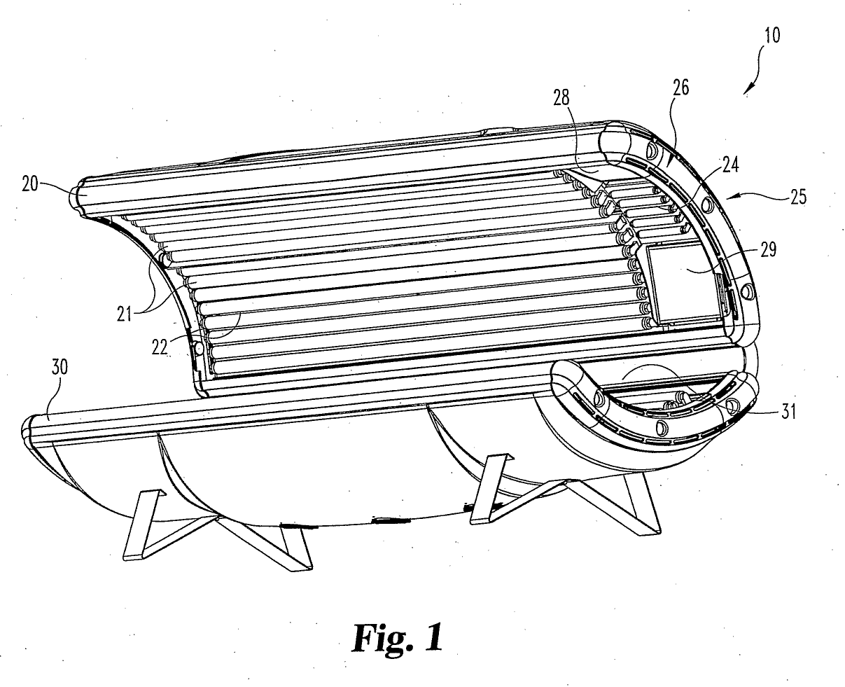

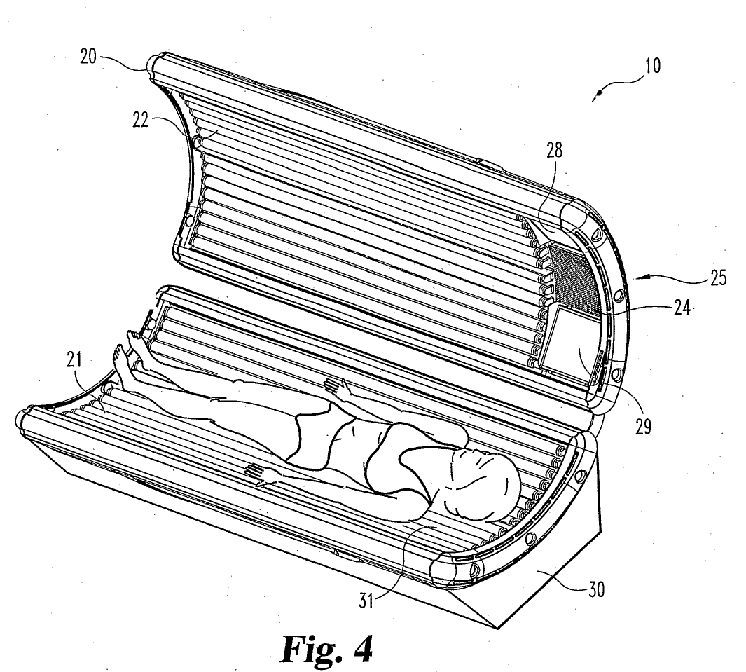

Referring to FIGS. 1-4, a tanning system, such as bed 10, according to one embodiment of the present invention is illustrated. Bed 10 incorporates one standard style of a tanning bed which includes a lower or base unit 30, sometimes called a bench, and a canopy or upper unit 20. Within each of the base unit 30 and canopy 20 are typically a plurality of parallel, elongate, tubular bulbs or lamps 21. Lamps 21 are preferably fluorescent ultraviolet producing lamps that contain mercury vapor. The tanning system may contain at least 8 ultraviolet lamps to provide ultraviolet illumination for tanning the body of a person, such as in a canopy alone, but preferably the bed 10 contains at least 24 or 32 lamps in the combined bench and canopy for providing an even exposure to the user's body.

Base unit 30 defines a support or bed surface 31 upon which a person lies during the application process. The support surface is often an acrylic plastic transparent panel. The person to be tanned typically lies in an orientation facing the canopy with their back to the support surface. Additionally, bulbs 21 may be separated from the user by ultraviolet (UV) transparent shields 22 in base unit 30 and canopy 20, or alternatively, for example, by wire mesh grids (not shown) in canopy 20.

Often the upper unit or canopy 20 is pivotally mounted to base unit 30 to allow the user ease of access to enter and lie upon support surface 31. To place the system in an operating position, the canopy is closed to a selected distance defining a gap or volume between the base unit 30 and canopy 20. The person having a face to be tanned is oriented in this gap.

A facial area 25 is preferably defined upon the canopy unit 20 and is oriented before the face of the person to be tanned. For ease of reference, the facial area is referred to as oriented “before” or facing the face of the person to be tanned which is defined with respect to the person and not an absolute direction. The area of the face referred to is generally the front portion extending between the person's neck and scalp, between the person's ears.

FIGS. 1-4 further show one or more devices 24 illustrating one embodiment of the present invention. By way of non-limiting example, devices 24 may alternatively be light sources having narrow band filters, lasers, low-pressure fluorescent lights, and/or gas discharge lights containing neon, sodium, argon, helium, xenon, and/or krypton. In the preferred embodiment, devices 24 collectively produce at least 500 lux to provide sufficient illumination to the skin of the user to produce the desired effects. Preferably the devices 24 will produce at least 1000 lux and more preferably at least 3000 lux.

In a preferred embodiment, devices 24 are an array 26 of at least 30 light emitting diodes operating to provide visible illumination to the face of the person to be tanned. In an alternate embodiment, devices 24 include 128 light emitting diodes, and may further include 300 light emitting diodes. In a further embodiment, devices 24 comprise three different types of LEDs, each type with a different principal wavelength and separate controller. The pulsing of each one of the types of LEDs begins at a different time than the pulsing of the other types of LEDs. With a pulse rate of about 15 Hz, from a distance it can appear that the color of the composite array changes. Preferably the pulse rate will be between 1 and 300 Hz, more preferably between and 100 Hz, and most preferably about 70 Hz. In an alternate embodiment, the pulse rate may be between 5 and 25 Hz.

In a preferred embodiment said array 26 produces an illumination power of at least 2 milliwatts per square centimeter. In a further preferred embodiment, said array 26 produces an illumination power of at least 3 milliwatts per square centimeter.

One or more specialized facial tanning units, for example 28 and 29, may be placed in the facial area 25 on the canopy 20. Facial tanning units 28 and 29 are configured within the facial area 25 upon the canopy 20 so that when the bed 10 is in its closed and operable position the facial tanning units 28 and 29 are directed at the face of the person to be tanned. Units 28 and 29 may contain high pressure, non-fluorescent lamps containing mercury vapor, and may have a filter to reduce UVC and optical light output and otherwise to produce a desired amount of UVA and UVB as is conventional in many prior art face tanners.

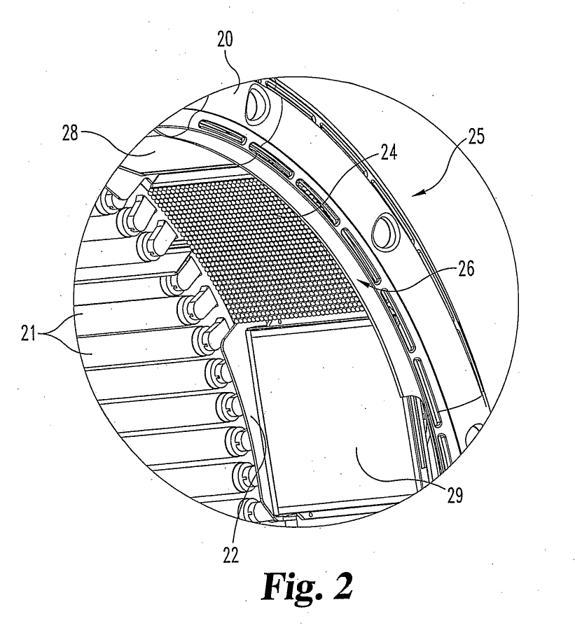

Shown in detail in FIG. 2 is an array 26 of one or more devices 24 emitting phototherapy light. Devices 24 are configured within the facial area 25 upon the canopy 20 so that when the bed 10 is in its closed and operable position the devices 24 are directed at the face of the person to be tanned. In a preferred embodiment devices 24 include a first type of light emitting diode with a center frequency of from about 580 to about 590 nm, a second type of light emitting diode with a center frequency of from about 622 to 642 nm and a third type of light emitting diode with a center frequency of from about 655 to 665 nm. Most preferably, the center frequencies are a subset of 525, 590, 632, 660, 680, 730, 880, and 930 nm. It shall be appreciated that while the most preferable frequencies for delivering skin rejuvenating effects have been described, conveniently available light sources having similar or substantially similar center frequencies may be substituted based upon commercial availability.

In a further preferred embodiment the light emitting diodes are pulsed. In a still further preferred embodiment the duty cycle of the pulses is 51% or less, and preferably greater than 0.001%. More preferably the duty cycles are about 30%. It may also be desirable to distribute devices 24 throughout the tanning system in order to expose various other parts of the body to skin rejuvenating light. For example, devices 24 may provide phototherapy light to the legs, stomach, or back, to name just a few non-limiting examples, in order to provide treatment for other known skin problems or promote skin wellness.

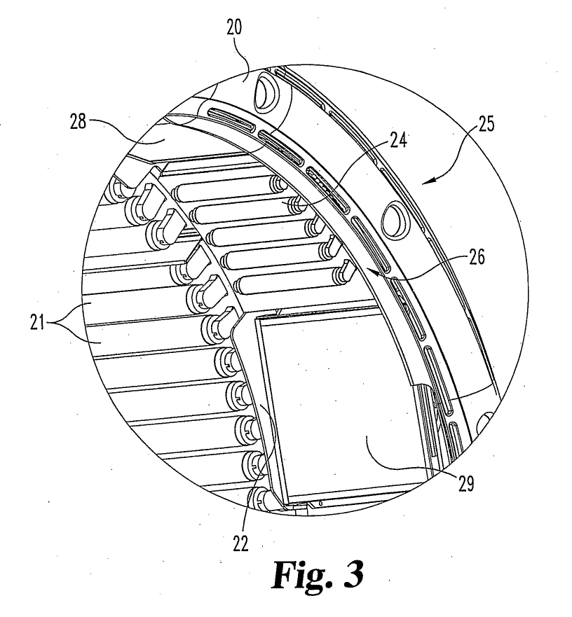

In the illustrated embodiment, for example, devices 24 are shown as an array 26 of LEDs configured to emit selected wavelengths of phototherapy light. Devices 24 are arranged in the center of the facial area 25 of canopy unit 20 such that the front of the devices 24 point towards the face of the person to be tanned. Devices 24 could be arranged in many other patterns not limited to an array. Arranged adjacent the devices 24 are facial tanners 28 and 29. Many other arrangements of one or more devices 24 and one or more face tanners within the facial area 25 are contemplated; for example, devices 24 may be low pressure fluorescent tubes as shown by FIG. 3. Other embodiments may include, by way of non-limiting example, a circular face tanner surrounded by a plurality of devices 24, or a grid pattern containing alternating squares of facial tanners and devices 24.

In a preferred embodiment, devices 24 are pulsed at a frequency between 1 and 200 Hz and have a duration of between one nanosecond and one second. More preferably the frequency of the pulses is about 50 Hz. One example of the duration of the pulses can be about 2 milliseconds.

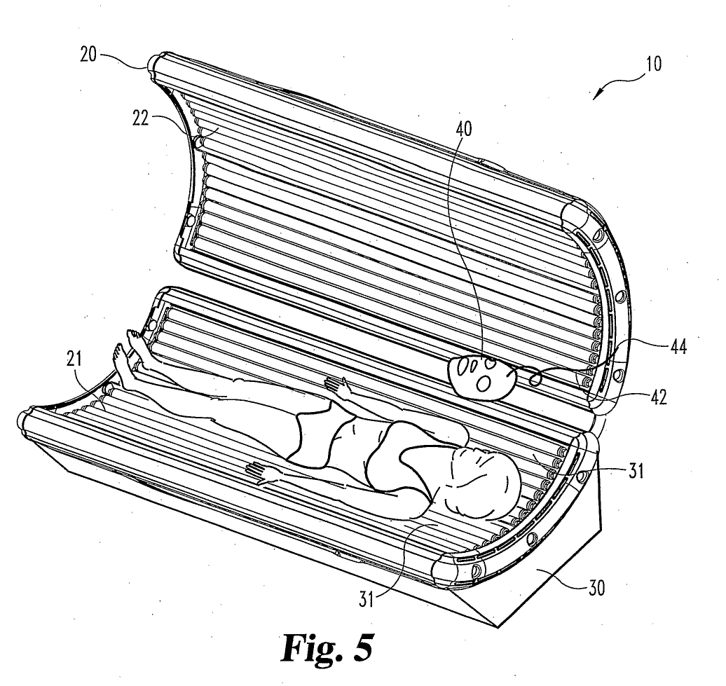

Turning to FIG. 5, an alternate embodiment of a tanning system 10 is illustrated. Tanning system 10 includes many of the features contained in the tanning system of FIG. 1, such as base 30, canopy 20, and bulbs 21, but replaces devices 24 with a face mask 40. Face mask 40 may resemble a face and otherwise be formed to be oriented on the face of a person to be tanned during operation of tanning system 10. Face mask 40 preferable includes a plurality of devices, similar to device 24 of FIGS. 1-4 for illuminating the face of the person to be tanned. In the preferred embodiment, face mask 40 provides non-UV illumination but may alternatively provide ultraviolet illumination, or a combination of both in further embodiments. Such illumination may be provided using a variety of light sources described herein with respect to devices 24. In the illustrated embodiment, face mask 40 is connected to canopy 24 at attachment point 44 through wire 42. It shall be understood that face mask 40 may alternatively be attached to base unit 30 or operate as stand alone device. In the illustrated embodiment, both power and control signals are delivered to face mask 40 through wire 42.

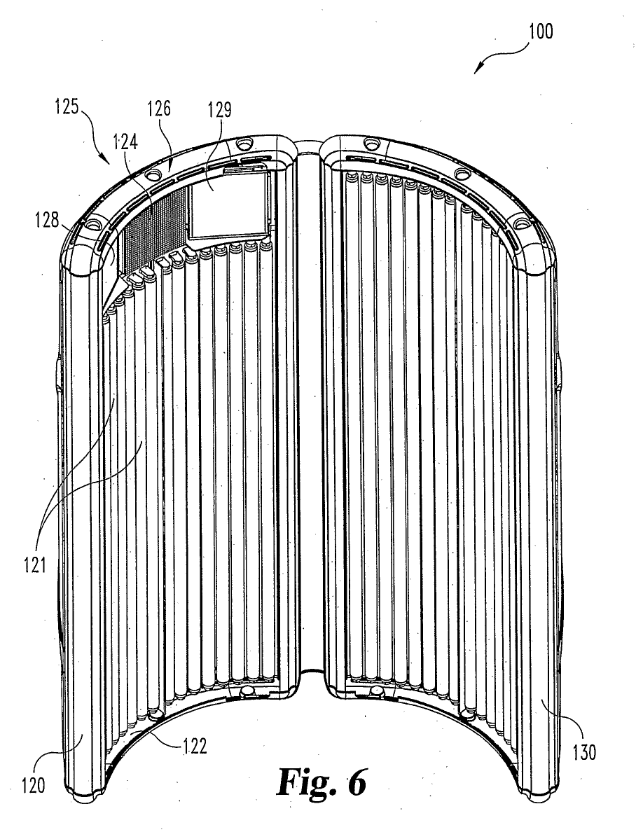

In an alternate embodiment, illustrated in FIG. 6, a tanning booth 100 is shown. Booth 100 includes a first or “back” portion 130 and a second or “front” portion 120 hingedly mounted to back portion 130. In an alternative arrangement (not shown) front portion 120 may be divided into two portions hinged to opposite sides of back portion 130. Booth 100 is moveable from an open position for ease of access to a closed or “operating” position. When booth 100 is in the operating position, a gap or volume is defined between back portion 130 and front portion 120. A person with a face to be tanned preferably stands within this volume during a tanning session and is substantially encircled by the booth 100. Mounted within back portion 130 and front portion 120 are a plurality of elongate, tubular tanning bulbs or lamps 121. Lamps 121 are separated from the user by UV transparent shields 122, or alternatively wire mesh grids (not shown) which are mounted to the front portion 120 and back portion 130.

In one preferred embodiment, the tanning booth 100 defines a facial area 125 before the face of the person to be tanned. The facial area 125 includes one or more ultraviolet facial tanners, for example 128 and 129, combined with one or more devices 124 emitting phototherapy light, predominantly similar to devices 24. In an alternate embodiment, the facial area 125 is mounted on adjustable tracks or an arm to allow the position of facial area 125 to be optimized for a particular user's height. Preferably, devices 124 are separated from the user with a minimum distance and/or an acrylic cover or mesh grid (not shown).

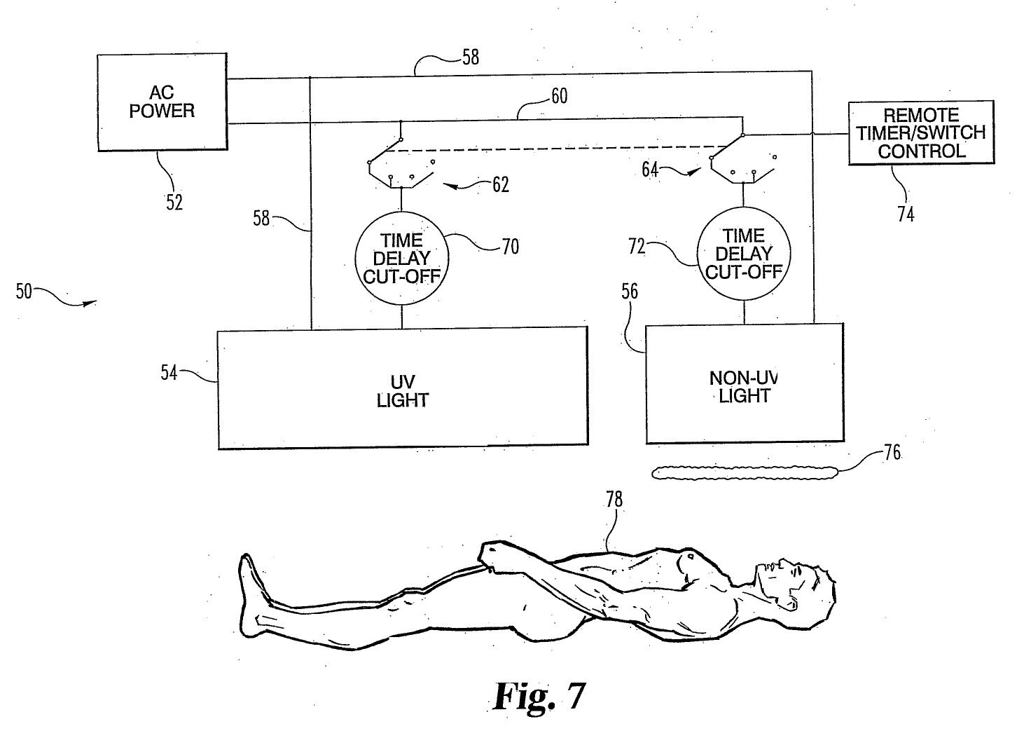

In a further embodiment, illustrated in FIG. 7, a tanning bed control system suitable for use with a tanning system providing phototherapy light is shown. Control system 50 includes AC power source 52, ultraviolet light source 54 and non-ultraviolet light source 56. Ultraviolet light source 54 and non-ultraviolet light source 56 are positioned to be above the body of the user 78 and the face of the user 78 respectively when the user 78 is properly oriented in the system. UV light source 54 is electrically connected to one pole of AC power source 52 through electrical wire 58 and to the other pole through electrical wire 60 and electrical switch 62. In a similar fashion, non-UV light source 56 is electrically connected to one pole of AC power source 52 through electrical wire 58 and to the other pole through electrical wire 60 and electrical switch 64. Electrical switches 62 and 64 are arranged to function as a double pole, four throw gang switch assembly. Thus, switch 64 will be forced to be in the same position as switch 62. As alternatives, two separately operable single pole switches could be used or electronic equivalents implemented. The following state transition table illustrates the desired combinations which can be achieved using this configuration:

| Switch 62 | Switch 64 | UV Light | Non-UV Light |
|---|---|---|---|
| A | A | ON | ON |
| B | B | ON | OFF |
| C | C | OFF | ON |
| D | D | OFF | OFF |

As illustrated in the state transition table, the user can configure the system to provide UV and non-UV light in combination, only UV light or only non-UV light. Additionally, the user may turn both light sources off. Using this control system, the tanning system remains functional to those users who may not want non-UV treatment, or to those who want only non-UV treatment.

In a still further preferred embodiment, control system 50 may include time delay cutoff circuits 70 and 72 connected in series with the electrical connection to UV light source 54 and non-UV light source 56 respectively. Preferably, circuit 70 limits the operation of UV light source 54 to a maximum twenty (20) minute interval and circuit 72 also limits the operation of non-UV light source 56 to a maximum twenty (20) minute interval.

Additionally, control system 50 may include a remote timer/switch control 74 for controlling the operation of switches 62 and 64. For example, remote timer 74 may be a system, implemented in software or hardware, for controlling the features of the associated tanning system in accordance with the desires of a particular user. Control system 50 may also have an emergency shut off switch, located in close proximity to the tanning system. Furthermore, non-UV light 56 may have associated with it a narrowband control filter 76 for selecting the particular bands of non-UV light to be delivered to the skin of the user 78.

While the invention is illustrated in the preferred context of a tanning bed and tanning booth, the concepts of many of the patent claims also encompass facial tanners, tanning canopies, and other devices which emit tanning light.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

1. A hybrid system for tanning a person and providing visible light to the person's face comprising:
a first panel of at least 8 ultraviolet fluorescent mercury vapor discharge lamps for tanning the body of a person; and
a non-UV light source that emits about 500 lux, said source being integrated with the system to be positionable near the face of the person to be tanned.

2. The system of claim 1 wherein said light source includes a first type of device emitting narrow band light.

3. The system of claim 1 wherein said light source emits at least about 1000 lux.

4. (canceled)

5. The system of claim 3 wherein said light source emits at least about 5000 lux.

6-7. (canceled)

8. The system of claim 1 in which said light source emits light having two different principal wavelengths.

9. The system of claim 8 in which said light source emits light having three different principal wavelengths.

10. The system of claim 1 in which said light source includes a first device emitting light having a principal wavelength in a range selected from the group consisting of 405-450 nm, 515-535 nm, 580-600 nm, 613-643 nm, 650-669 nm, 670-690 nm, 720-740 nm, 870-890 nm, and 920-940 nm.

11. (canceled)

12. The system of claim 10 in which said light source includes a second device emitting light having a principal wavelength different from said principal wavelength of said first device in a range selected from the group consisting of 405-450 r, 515-535 nm, 580-600 mm, 613-643 nm, 650-669 nm, 670-690 nm, 720-740 nm, 870-890 nm, and 920-940 nm.

13. (canceled)

14. The system of claim 12 in which said light source includes a third device emitting light having a principal wavelength different from said principal wavelength of said first device and said principal wavelength of said second device in a range selected from the group consisting of 405-450 nm, 515-535 nm, 580-600 nm, 613-643 nm, 650-669 nm, 670-690 nm, 720-740 nm, 870-890 n, and 920-940 nm.

15. (canceled)

16. The system of claim 14 in which said first device emits light having a principal wavelength from about 580 to about 600 nm, said second device emits light having a principal wavelength from about 622 to 642 nm, and said third device emits light having a principal wavelength from about 650 to 670 nm.

17. (canceled)

18. The system of claim 1 which additionally comprises an ultraviolet face tanning lamp positioned adjacent to said light source.

19-20. (canceled)

21. The system of claim 1 in which at least some of said devices are pulsed.

22. The system of claim 21 in which all of said devices are pulsed.

23. The system of claim 21 in which said light source includes two devices each emitting light having a different frequency.

24. The system of claim 23 in which the pulsing of devices at one frequency occur at a different timing than the pulsing of said devices at the other frequency.

25. The system of claim 24 in which the duty cycle of said pulses is less than 51%.

26. The system of claim 25 in which the pulses for the devices of said first frequency occur only when the devices of the second frequency are not pulsing.

27. (canceled)

28. The system of claim 21 in which the duty cycle of the pulses are near 20%.

29. The system of claim 21 in which the frequency of the pulses are between 1 and 300 Hz.

30. The system of claim 29 in which the frequency of the pulses are between 30 and 70 Hz.

31. (canceled)

32. The system of claim 21 in which the duration of the pulses are between one microsecond and 500 milliseconds.

33. The system of claim 1 in which said plurality of devices includes at least 30 devices.

34. (canceled)

35. The system of claim 33 in which said plurality of devices includes at least 100 devices.

36-38. (canceled)

39. The system of claim 35 in which the duration of the pulses are near 2 milliseconds at 50 Hz.

40. The tanning system of claim 1 in which said system is a tanning bed, a tanning booth, or a canopy tanner.

41-42. (canceled)

43. The tanning system of claim 1 in which said plurality of devices produces an illumination power of at least 0.5 milliwatts per square centimeter.

44. (canceled)

45. The tanning system of claim 43 in which said plurality of devices produces an illumination power of at least 3 milliwatts per square centimeter.

46. (canceled)

47. The tanning system of claim 1 in which one of said devices is a light emitting diode.

48. The tanning system of claim 47 in which all of said devices are light emitting diodes.

49. The tanning system of claim 1 in which one of said devices is a laser.

50. The tanning system of claim 1 in which one of said devices is a fluorescent light having narrowband filters.

51. The tanning system of claim 50 additionally comprising a filter to further narrow the bandwidth.

52. The tanning system of claim 1 in which one of said devices is a gas discharge lamp.

53. The tanning system of claim 52 in which said gas discharge lamp contains an element selected from the group consisting of neon, sodium, helium, krypton, xenon, mercury, and argon.

54-55. (canceled)

56. The tanning system of claim 1 in which said light source is in the shape of a facemask.

57. The tanning system of claim 56 in which said facemask is connected to said first panel.

58. A system for tanning a person comprising:
a first panel of at least 8 devices emitting ultraviolet light for tanning the body of a user;
a non-UV light source for providing phototherapy light to the body of a user connected to said first panel; and
a selective controller operably connected to said first panel and said light source for allowing a user to operate said system in a plurality of modes comprising:
a tanning only mode wherein said first panel is enabled and said light source is disabled;
a combination mode wherein said first panel is enabled and said light source is enabled.

59. The system of claim 58 wherein said plurality of modes further comprises:
a phototherapy only mode wherein said first panel is disabled and said light source is enabled.

60. The system of claim 58 wherein said selective controller uses a ganged switch so that modes can be selected with a single control device.

61. The system of claim 58 wherein said first panel is mounted to a housing and said selective controller is mounted to said housing.

62. The system of claim 58 wherein said selective controller is housed in a control box which is separate from said panel.

63-67. (canceled)

68. The system of claim 58 wherein said devices emitting ultraviolet light are mercury vapor discharge lamps.

69. The system of claim 58 wherein said non-UV light source includes at least 30 light emitting diodes.

70. The tanning system of claim 58 in which said system is a tanning bed, a tanning booth, or a canopy tanner.

71-72. (canceled)

73. The tanning system of claim 58 in which said non-UV light source is a facemask.

74. (canceled)

75. The system of claim 58 further comprising:
a second panel having at least one device emitting ultraviolet light positioned near the face of the person to be tanned;
wherein said selective controller is further operable to allow a user to control said second panel.

76. A hybrid system for tanning a person and providing visible light to the person's face comprising:
a first panel of at least 8 ultraviolet fluorescent mercury vapor discharge lamps for tanning the body of a person; and
a facemask that emits at least 500 lux, said source being integrated with the system to be positionable near the face of the person to be tanned.

77. The system of claim 76 in which said facemask emits ultraviolet light.

78. The system of claim 76 in which said facemask emits non-UV light.

79. The system of claim 78 in which said facemask emits narrow band light.

80. (canceled)

81. The system of claim 76 in which said facemask includes at least 25 LEDs.

82-84. (canceled)

85. The system of claim 81 in which said facemask includes at least 1000 LEDs.

86-90. (canceled)
